(19) United States
(12) Patent Application Publication
(10) Pub. No.: US 2026/0091169 A1
(43) Pub. Date: Apr. 2, 2026

(54) PUMP DEVICE AND ASSOCIATED METHOD

(71) Applicant: Evanesce Medical, Inc., Excelsior, MN (US)

(72) Inventors: Ishmael BENTLEY, Edina, MN (US); Jeffrey PETERS, Excelsior, MN (US); Patrick Jan BOLDENOW, Bloomington, MN (US); Lawrence WALES, Maplewood, MN (US)

(73) Assignee: Evanesce Medical, Inc., Excelsior, MN (US)

(21) Appl. No.: 19/346,169

(22) Filed: Sep. 30, 2025

Related U.S. Application Data
(60) Provisional application No. 63/701,907, filed on Oct. 1, 2024.

(51) Int. Cl. A61M 5/145; A61M 5/168

(52) U.S. Cl. CPC A61M 5/1452; A61M 5/16804

(57) ABSTRACT

A pump device including: a chamber disposed in a body portion; a plunger disposed in the chamber and configured to move within the interior of the chamber to deliver medication in an interior of the body portion through an outlet of the body portion, the plunger including a receiving portion; a retainer disposed within the body portion, which includes a plurality of rod openings; a plurality of rods removably retained within the rod openings; and a button disposed in the body portion, which includes a pushing surface and a protrusion, the button configured, when the pushing surface is pushed, to move within the body portion such that the protrusion of the button contacts a first rod of the plurality of rods in the plurality of rod openings and moves the first rod out of the first rod opening and into the receiving portion of the plunger.

### CROSS REFERENCE TO RELATED APPLICATION

This application claims the benefit of U.S. Provisional Patent Application No. 63/701,907, filed Oct. 1, 2024, the entire contents of which are incorporated herein by reference.

### FIELD

The present disclosure is directed to a pump device and associated method, and more specifically, to a pump device and associated method for delivering multiple doses of a medication.

### SUMMARY

In some examples, the present invention provides a pump device, and/or an associated method, such as a method of using the pump device.

In some examples, a pump device configured to deliver a medication, includes: a body portion; a chamber disposed in the body portion, the chamber including an interior and an outlet, the interior configured to hold the medication and to deliver the medication through the outlet; a plunger disposed in the chamber and configured to move within the interior of the chamber to deliver the medication in the interior through the outlet, the plunger including a receiving portion; a retainer disposed within the body portion, the retainer including a plurality of rod openings; a plurality of rods removably retained within the plurality of rod openings; and a button disposed in the body portion, the button including a pushing surface and a protrusion, the button configured, when the pushing surface of the button is pushed, to move within the body portion such that the protrusion of the button is brought into contact with a first rod of the plurality of rods in a first rod opening in the plurality of rod openings and to move the first rod out of the first rod opening and into the receiving portion of the plunger.

In some examples, the retainer is configured to rotate to align a second rod of the plurality of rods in a second rod opening of the plurality of rod openings with the first rod in the receiving portion of the plunger.

In some examples, the retainer is configured to rotate to align a second rod of the plurality of rods in a second rod opening of the plurality of rod openings with the first rod in the receiving portion of the plunger, and the button is configured, when the pushing surface of the button is pushed, to move within the body portion such that the protrusion of the button is brought into contact with the second rod and to move the second rod out of the second rod opening and into the receiving portion of the plunger, thereby delivery a first dose of the medication from the interior of the chamber through the outlet of the chamber.

In some examples, the body portion includes an upper body portion and a lower body portion coupled to one another, the chamber is disposed within the lower body portion, and the button is disposed in the upper body portion.

In some examples, the retainer is configured to rotate in only one direction.

In some examples, at least one of the protrusion of the button and the plurality of rods comprises a magnet, and the other one of the protrusion and the plurality of rods comprises a magnetic material, such that the protrusion and one rod of the plurality of rods are magnetically attracted to one another when the one rod of the plurality of rods is aligned with the protrusion.

In some examples, the protrusion of the button is configured to slide one rod of the plurality of rods from the retainer into a receiving portion of the plunger.

In some examples, the pump device has a bottom end and a top end opposite the bottom end, wherein the chamber is disposed closer to the bottom end than the top end, and the retainer is configured to rotate in a clockwise direction, when viewed from the top end of the pump device, to align a second rod of the plurality of rods in a second rod opening of the plurality of rod openings with the first rod in the receiving portion of the plunger.

In some examples, a pump device configured to deliver a medication, includes: a body portion; a chamber disposed in the body portion, the chamber including an interior and an outlet, the interior configured to hold the medication and to deliver the medication through the outlet; a plunger disposed in the chamber and configured to move within the interior of the chamber to deliver the medication in the interior through the outlet, the plunger including a receiving portion; a retainer disposed within the body portion; a plurality of rods removably retained within the retainer; and a mechanism configured to move the plunger within the chamber thereby delivery a dose of medication from the chamber, wherein the mechanism is configured to engage with at least one rod of the plurality of rods, thereby moving the plunger within the chamber.

### BRIEF DESCRIPTION OF THE FIGURES

The accompanying drawings, which are incorporated into and constitute a part of this specification, illustrate various exemplary embodiments and, together with the description, serve to explain the principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

The drawings illustrate example embodiments of the present disclosure and, along with the description, serve to explain the principles of the disclosure. The drawings are only illustrative of certain embodiments and do not limit the disclosure or invention.

FIG. 1 is an isometric view of the pump device, according to some embodiments.

FIG. 2 is an exploded isometric view of the pump device of FIG. 1, according to some embodiments.

FIG. 3 is an isometric view of the pump device of FIG. 1 illustrating some of the components of the pump device in cross section, the pump device in a first configuration, according to some embodiments.

FIG. 4 is an isometric view of the pump device of FIG. 1 illustrating some of the components of the pump device in cross section, the pump device in a second configuration, according to some embodiments.

FIG. 5 is an isometric view of the pump device of FIG. 1 illustrating some of the components of the pump device in cross section, the pump device in a third configuration, according to some embodiments.

FIG. 6 is an isometric view of the pump device of FIG. 1 illustrating some of the components of the pump device in cross section, the pump device in a fourth configuration, according to some embodiments.

FIG. 7 is an isometric view of the pump device of FIG. 1 illustrating some of the components of the pump device in cross section, the pump device in a fifth configuration, according to some embodiments.

FIG. 8A is a top view of showing some of the components of the pump device of FIG. 1, according to some embodiments.

FIG. 8B is a side view showing some of the components of the pump device of FIG. 1, according to some embodiments.

### DETAILED DESCRIPTION

There are many embodiments described and illustrated herein. The described embodiments are neither limited to any single aspect nor implementation thereof, nor to any combinations and/or permutations of such aspects and/or implementations. Moreover, each of the aspects of the described embodiments, and/or implementations thereof, may be employed alone or in combination with one or more of the other aspects of the described embodiments and/or implementations thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example”embodiment(s).

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal. ” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

The term “distal end,” or any variation thereof, refers to the portion of a device farthest from an operator of the device during a procedure. Conversely, the term “proximal end,” or any variation thereof, refers to the portion of the device closest to the operator of the device. Further, any use of the terms “around,” “about,” “substantially,” and “approximately”generally mean +/−10% of the indicated value(s).

As discussed herein, a medication delivery device may include a housing with a chamber for storing and dispensing medication. A plunger within the chamber may move to deliver medication through an outlet. A rotating retainer with multiple rod openings may house a plurality of rods. A button or other activation device, when pushed, may move a rod from the cylinder into the plunger, activating medication delivery.

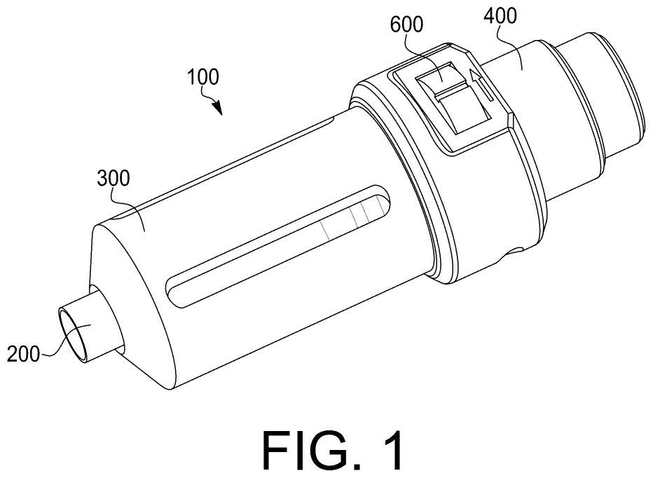

With reference to the drawings, FIG. 1-7 illustrate a pump device 100, according to some embodiments. As the figures show, the pump device 100 may include a chamber 200. In some embodiments, the pump device 100 is wearable, such that the pump device 100 is configured, intended, and otherwise suitable for wearing. The pump device 100 may be attached, secured, or otherwise worn by a user using, for example, one or more securing straps (e.g., using a Velcro™ straps, adhesive materials or patches, belt(s) such as including but not limited to elastic bands, belts, straps, or the like, etc.). In some instances, the securing straps may connect to and/or surround at least a portion of the pump device 100, as well as connect to and/or surround at least a portion of the body of the wearer/user, thereby to removably secure the pump device 100 to the user. The pump device 100 may be worn such that components of the pump device 100 discussed herein to operate the pump device 100 may be accessible by a user while the pump device 100 is worn, without requiring removal of the pump device 100 from a user's body to perform such operations. The pump device 100 may be detachable such that a user may detach the pump device 100 and may re-attach (e.g., wear) the pump device 100 after the detaching.

The chamber 200 may be configured to hold a medication, which is to be delivered by the pump device 100 to a patient, as further described. In some embodiments, the chamber 200 is an expandable chamber.

As the figures illustrate, the chamber 200 may include a body 201 defining an interior configured to hold the medication, and an outlet 203 through which the medication is to be delivered from the interior of the body 201, such as to the patient. A plunger 205 may be disposed within the interior of the body 201. The plunger 205 may be configured to move within the chamber 200 toward the outlet 203, to deliver the medication from the interior of the body 201 through the outlet 203. The plunger 205 may include receiving portion 207 which is used to move the plunger 205 to deliver a specified dose of the medication to the patient, as discussed further below. In some embodiments, the pump device 100 is prepared, such as by filling the chamber 200 with the medication, at any location, such as but not limited to a clinic, hospital, emergency room, urgent care center, doctor's office, pharmacy, emergency vehicle such as an ambulance, or at a patient's residence.

The outlet 203 of the chamber 200 may include one or more threads configured to connect threadingly with one or more corresponding threads of another component, such as a catheter or a needle inserted into the patient, for example. It will be understood that although a thread-based connection is discussed herein as an example, chamber 200 may connect to another component in any applicable manner such as, but not limited to, a friction based connection, a snap connection, a compression based connection, a coupling, etc. In some embodiments, the pump device 100 may be supplied with tubing, such as catheter (the catheter tubing having a length of approximately, for example, 4 inches, 5 inches, 6 inches, 7 inches, 8 inches, or more than 8 inches). In some embodiments, the pump device 100 is removable from and reattachable to the catheter or needle. In some embodiments, the pump device 100 is irremovable from the catheter or needle. In some embodiments, the pump device 100 delivers medication to a patient, or an intermediate device or system that is between the pump device 100 and the patient. In some embodiments, the outlet 203 is configured to prevent removal and reattachment of the other component (e.g., the catheter or the needle), thereby preventing contamination resulting from such removal and reattachment. In some embodiments, an intermediate component may be provided between the outlet 203 and the other component (e.g., the catheter or needle), the other component being a sterile component, such as a sterile component intended to be replaced after removal of the other component from, and before reattachment of the other component to, the pump device 100.

In some embodiments, the chamber 200 may be configured to hold approximately 1 ml to approximately 50 ml or more of medication such as, for example, 1 ml, 2 ml, 3 ml, 4 ml, 5 ml, 6 ml, 7 ml, 8 ml, 9 ml, 10 ml, 15 ml, 20 ml, 25 ml, 30 ml, 35 ml, 40 ml, 45 ml, 50 ml, or more than 50 ml of the medication. As further discussed, the pump device 100 may be configured to deliver multiple doses (e.g., 2 doses, 3 doses, 4 doses, 5 doses, 6 doses, 7 doses, 8 doses, 9 doses, 10 doses, or more than 10 doses) of the medication. In some embodiments, individual doses may be approximately, for example, 0.5 ml, 1 ml, 1.5 ml, 2 ml, 2.5 ml, 3 ml, 3.5 ml, 4 ml, 4.5 ml, 5 ml, or more than 5 ml. According to some embodiments, a total volume of the chamber 200 may not exceed a maximum recommended dosage of the medication stored in the chamber 200.

The pump device 100 may include a lower body portion 300 and an upper body portion 400, which form a housing which retains other components of the pump device 100. The lower body portion 300 may include an interior sized and shaped to hold the chamber 200 with the medication. The lower body portion 300 may be one piece (e.g., a unitary body), or may be more than one piece, such as two pieces that may be snap-fit or otherwise attached to one another, thereby forming the lower body portion 300. In some embodiments, the lower body portion 300 may be formed by snap-fitting together a hinged body. The lower body portion 300 may be a plastic, metal, composite, or other applicable material.

As the figures show, in some embodiments, the lower body portion 300 may include one or more openings 301 (e.g., a window), through which one may visually observe an amount of the medication within the interior of the body 201. In some embodiments, the openings 301 may include a fully or partially transparent material (e.g., plastic or glass), for example, through which the medication may be observed. Thus, in some embodiments, it may be observable how much of the medication initially within the chamber 200 has been delivered, and/or how much of the medication remains within the chamber 200.

The upper body portion 400 may be sized and shaped to retain the lower body portion 300, as well as other components of the pump device 100 as further described. For example, as shown in the figures, the upper body portion 400 may include one or more channels configured to retain one or more corresponding protrusions of the lower body portion 300. The upper body portion 400 may be configured to prevent or impede removal of the lower body portion 300 from the pump device 100, once chamber 200 with the medication is placed within the lower body portion 300, and the lower body portion 300 is assembled with the upper body portion 400.

The upper body portion 400 may be more than one piece, such as two pieces that may be snap-fit or otherwise attached to one another to form the upper body portion 400. The upper body portion 400 may include structure to prevent or impede disassembly of pieces of the upper body portion 400 from one another, which may prevent or impede removal of the lower body portion 300 from the pump device 100, as well as access to or removal of other components of the pump device 100. In some embodiments, the upper body portion 400 may be formed by snap-fitting together a hinged body. The upper body portion 400 may be a plastic, metal, composite, or other applicable material.

The pump device 100 may include a first plate 500 within an interior of the upper body portion 400. The first plate 500 may include an opening 501, an axle 503, and a protrusion 505. The first plate 500 may be a plastic, metal, or other applicable material.

The pump device 100 may include a retainer 600 within the interior of the upper body portion 400. The retainer 600 may include one or more rod openings 601, and an axle opening 603. The retainer 600 may include 2, 3, 4, 5, 6, 7, or more than 7 rod openings 601. The axle 503 of the first plate may extend through the axle opening 603 of the retainer 600, such that the retainer 600 may rotate on the axle 503 of the first plate 500, as further discussed below. The retainer 600 may be a plastic, metal, or other applicable material. As the figures show, in some embodiments, the retainer 600 includes a cylinder that rotates. However, it is understood that the retainer 600 is not limited to a cylinder, and may include other shapes (e.g., a pentagon, hexagon, heptagon, octagon, or other shapes). In some embodiments, the retainer 600 does not rotate.

As the figures show, the upper body portion 400 may include an opening through which at least a portion of the retainer 600 extends. This opening in the upper body portion 400 may permit rotation of the retainer 600 for delivery of the doses of medication from the chamber 200, as further described.

The pump device 100 may include a plurality of rods 700 removably retained within the rod openings 601 of the retainer 600, such as by friction between the rods 700 and walls of the rod openings 601. In some embodiments, each rod openings 601 includes one rod 700. The rods 700 may be a plastic, metal, composites, or other applicable material. The metal material of the rods 700 may be a magnetic material (e.g., a metal material that is attracted to or repelled by a magnet), or may be a magnet (e.g., a metal material that produces a magnetic field). In some embodiments, the rods 700 may be relatively strong magnets, such as rare-earth magnets. In some embodiments, regardless of whether the rods 700 are or are not magnets or magnetic materials, the rods 700 may include features such that the rods 700 that engage with and retain connection with one another. In some embodiments, the engaging features may include barbs, protrusions, indentations, or other features. In some embodiments, an exterior of the rods 700 and an interior of the rod openings 601 in the retainer 600 are correspondingly threaded, so that movement of one of the rods 700 from its rod opening 601 results in rotation of the retainer 600, such that a subsequent rod openings 601 is aligned for movement of the next one of the rods 700 from its rod opening 601, as further described.

In some embodiments, when the rods 700 are magnets, one or more other components of the pump device 100 may be non-magnets or nonmagnetic materials, including nonmagnetic metals, thereby preventing the rods 700 from being attracted to these other components. Thus, for example, in some embodiments, the rods 700 may be magnets, and the retainer 600 may be a nonmagnetic material.

The pump device 100 may include a second plate 800 within the interior of the upper body portion 400. The second plate 800 may include an opening 801, an axle opening 803, and recess 805. As the figures show, the retainer 600 including the rods 700 may be disposed between the first plate 500 and the second plate 800, such that the axle 503 of the first plate 500 extends through the axle opening 603 of the retainer 600, and into the axle opening 803 of the second plate. The protrusion 505 of the first plate 500 may be disposed within the recess 805 of the second plate 800.

As the figures show, the opening 501 of the first plate 500 may be aligned with the opening 801 of the second plate 800, and the retainer 600, when rotated, may align the rod opening 601 with the opening 501 and the opening 801. By this arrangement, each one of the rods 700 that is removable retained within the rod openings 601 of the retainer 600 may be delivered from the retainer 600 through the opening 501 of the first plate 500, as further discussed. In some embodiments, the opening 501 of the first plate 500 and the opening 801 of the second plate 800 may be aligned along a centerline of the pump device 100, such that when one of the rods 700 removable retained within the rod opening 601 of the retainer 600 is aligned with the opening 501 of the first plate 500, the rod 700 also is aligned with the centerline of the pump device 100. Thus, in some embodiments, an axis of rotation around which the retainer 600 rotates—e.g., the axle 503 of the first plate 500—is offset from the centerline of the pump device 100.

Further, after delivery of the rod 700 from the retainer 600 through the opening 501 of the first plate 500, the retainer 600 may be rotated further, such that the rod opening 601 from which the rod 700 was delivered is no longer aligned with the opening 501 of the first plate, but such that another rod opening 601 that includes another one of the rods 700 is aligned with the opening 501 of the first plate and the opening 801 of the second plate. This is further discussed below.

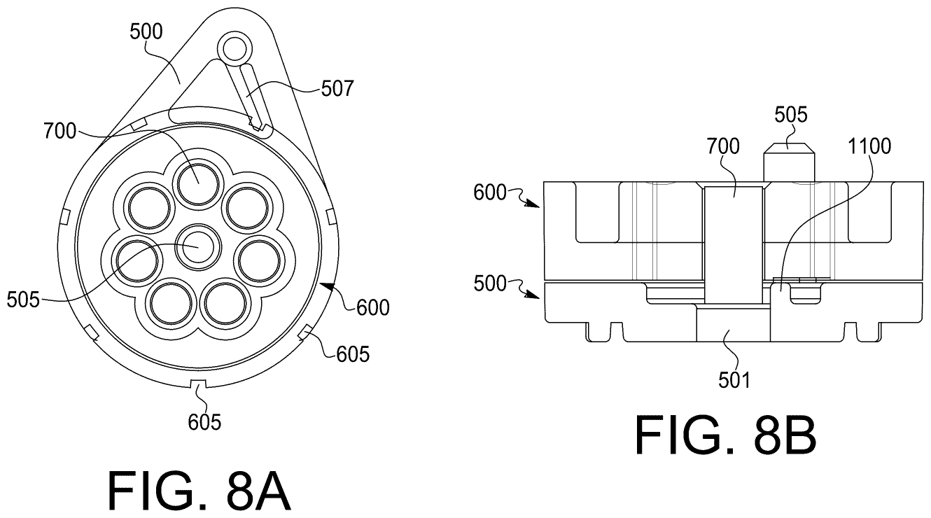

In some embodiments, the pump device 100 includes one or more components that prevent rotation of the retainer 600 until delivery from the retainer 600 of the rod 700 that is aligned with the opening 501 of the first plate 500 and the receiving portion 207 of the plunger 205, thereby preventing a given dose of the medication in the chamber 200 from being delivered. For example, with reference to FIGS. 8A and 8B, in some instances, a component 1100, such as but not limited to an arm, protrusion, tab, or other structure is brought into contact with the rod 700 that is aligned with the opening 501 of the first plate 500 and the receiving portion 207 of the plunger 205. The component 1100 may block rotation of the retainer 600 as long as the rod 700 that is aligned with the opening 501 of the first plate 500 and the receiving portion 207 of the plunger 205 is within the retainer 600, such as by the component 1100 contacting the rod 700. When the rod 700 that was aligned with the opening 501 of the first plate 500 and the receiving portion 207 of the plunger 205 is no longer within the retainer 600 (e.g., the rod 700 is delivered from the retainer 600), the component 1100 may permit rotation of the retainer 600. In some instances, the rod 700 that is aligned with the opening 501 of the first plate 500 and the receiving portion 207 of the plunger 205 must be fully delivered from the retainer 600, through the opening 501 of the first plate 500, before the retainer 600 may be rotated. Thus, this arrangement prevents delivery of less than a full dose of the medication before permitting rotation of the retainer 600 for later delivery of a subsequent dose. In some instances, the component 1100 may be a part of or otherwise connected to the first plate 500.

The pump device 100 may include a button 900, and a spring 1000, retained within the interior of the upper body portion 400. The button 900 may include a pushing surface 901, a retaining feature 903, and a protrusion 905. Pushing of the button 900 and corresponding operation of the pump device 100 are described below. In some embodiments, the pump device 100 may include bumps, ridges, protrusions, or other structure to prevent or at least inhibit inadvertent pressing of the button 900 and inadvertent operation of the pump device 100. In some embodiments, the pushing surface 901 may include a recess, thereby providing tactile feedback indicating that a user's finger is aligned with the pushing surface 901, which may be useful to a user with visual impairment, for example.

The retaining feature 903 of the button 900, which may be in the form one or more protrusions, may be retained within one or more corresponding channels in the upper body portion 400. By this arrangement, the button 900 may be prevented or impeded from being removed from the pump device 100. The button 900 may include a plastic, metal, or other applicable material. For example, in some embodiments, portions of the button 900 such as the pushing surface 901 may be plastic, while the protrusion 905 may be metal. In some embodiments, the protrusion 905 may be a magnetic material, such as a magnetic metal, or may be a magnet. Thus, the protrusion 905 that is a magnetic material or a magnet may attract or be attracted to the rods 700 that are magnets or a magnetic material, thereby aligning the protrusion 905 and the rods 700 with one another, such as during movement of the rods 700 from the retainer 600 as further described.

The spring 1000 may be disposed between the upper body portion 400 and the button 900, such that the spring biases the button 900 away from the upper body portion 400 and away from a direction in which the button is pushed to deliver the medication from the pump device 100, as described in further detail below.

In some embodiments, the retainer 600 is configured to rotate in only one direction. Thus, as shown, in some embodiments, the retainer 600 includes grooves 605 on an outer surface which interact with a ratcheting protrusion 507 of the first plate 500, the grooves 605 and ratcheting protrusion 507 sized, shaped, and oriented such that rotation of the retainer 600 is permitted in only one direction (e.g., clockwise as shown in the figures, when the pump device 100 is viewed from a top end thereof, the top end opposite a bottom end of the pump device 100, the chamber 200 being at the bottom end of the pump device 100). For example, in some embodiments, each of the grooves 605 in the retainer 600 include a first edge of approximately 90 degrees, which prevents rotation of the retainer 600 in one direction due to an interaction between the first edge and the ratcheting protrusion 507; and each of the grooves 605 in the retainer 600 include a second edge that is angled or chamfered, which permits rotation of the retainer 600 in one direction by allowing movement of the ratcheting protrusion 507 over the second edge. In some embodiments, geometry of the ratcheting protrusion 507 relative to the retainer 600 and/or the grooves 605 permits rotation of the retainer 600 in only one direction. In some embodiments, the retainer 600 incudes notations that are visible through the window in the upper body portion 400, the notations indicating the dose number (e.g., 1, 2, 3, 4, 5, 6, 7, etc.) and/or medication amount that is to be delivered or has been delivered, as further described.

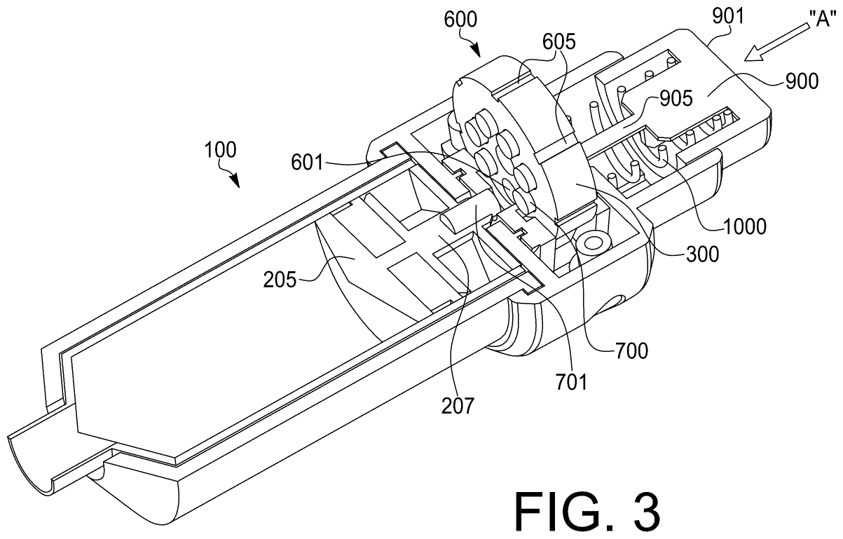

With reference to the figures, including FIG. 3-7, descriptions follow of actuation of the pump device 100 to deliver the doses of the medication from the chamber 200 through the outlet 203 of the chamber 200.

With reference to FIG. 3, the figure shows the first configuration in which no dose of the medication has been delivered by the pump device 100, and in which further rotation of the retainer 600 and pushing of the button 900 will deliver the first dose. In the first configuration, as FIG. 3 shows, a first one of the rods 700 (designated as first rod 701) from the retainer 600 has been delivered to the receiving portion 207 of the plunger 205, as described.

As shown, a force has been applied against the pushing surface 901 of the button 900, thereby moving the pushing surface 901 in a direction A, and correspondingly moving the protrusion 905 of the button 900 in the direction A. The movement of the button 900 was opposed by a force applied by the spring 1000 to the button 900, and thus it is understood that the button 900 was pushed with a force sufficient to overcome the force applied by the spring 1000.

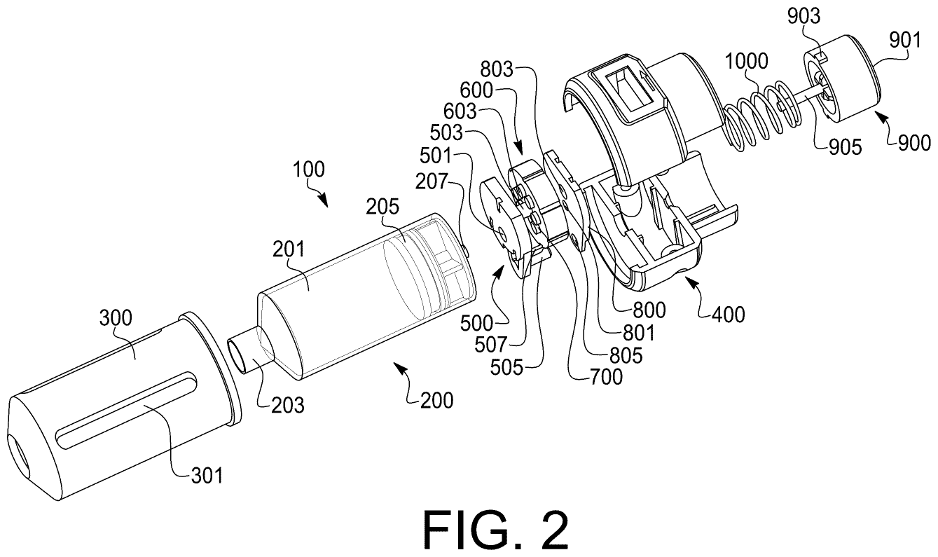

With reference to FIGS. 2 and 3, the protrusion 905 of the button 900 is aligned with the opening 801 of the second plate 800, which is aligned with the opening 501 of the first plate 500, such that when the button 900 was pushed, the protrusion 905 contacted the first rod 701 within the rod opening 601 of the retainer 600 aligned with the opening 501 of the first plate 500. The protrusion 905 of the button 900 is of a sufficient length, such that the first rod 701 was delivered from the rod opening 601 of the retainer 600, through the opening 501 of the first plate 500, and the first rod 701 was received by the receiving portion 207 of the plunger 205. As FIG. 3 shows, in some embodiments, when the first rod 701 is within the receiving portion 207 of the plunger 205, the plunger 205 has not moved within the body 201 of the chamber 200, and, thus, no medication has been delivered from the interior of the body 201 through the outlet 203. In some embodiments, the rods 700, the plunger 205, or the receiving portion 207 include features that prevent the first rod 701 from moving from its rod opening 601 when the receiving portion 207 is not disposed so as to be able to receive the first rod 701. If rods 700 are interconnecting rods, deploying a rod into empty space may be mitigated by positioning a base within a threshold proximity of the rods such that the first rod 701 connects to the base during deployment. As discussed above, in some embodiments, the protrusion 905 may be made of a metal material. The metal material may be a magnetic material (e.g., a metal material that is attracted to or repelled by a magnet), or may be a magnet (e.g., a metal material that produces a magnetic field), such that the protrusion 905 is attracted to the rods 700 and/or vice versa. The metal, magnet, or magnetic material of the protrusion 905 may help to align the protrusion 905 with the rods 700.

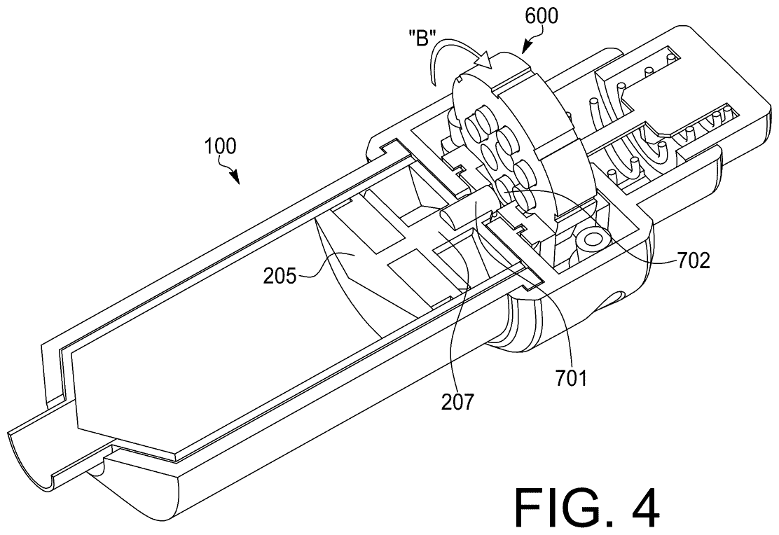

With reference to FIG. 4, the figure shows a second configuration in which no dose of the medication has been delivered by the pump device 100, but the pump device 100 is configured to deliver the first dose of the medication upon actuation of the button 900. As the figure show, the retainer 600 has been rotated on the axle 503 of the first plate 500 (see, e.g., FIG. 2), in a direction B. Thus, after rotation, as the figure shows, another rod opening 601 of the retainer 600, in which a second one of the rods 700 (designated as second rod 702) is removably retained, is aligned with the opening 501 of the first plate 500. In the configuration of FIG. 4, the pump device 100 is ready to provide, but has not yet provided, the first dose of the medication through the outlet 203 of the chamber 200.

As discussed, in some embodiments, each of the rods 700 are magnets. Thus, in some embodiments, in the configuration FIG. 4 shows, the attractive force between the first rod 701 already within the receiving portion 207 of the plunger 205 (see, e.g., FIG. 2) and the second rod 702 which is to be pushed into contact with the first rod 701 prevents or impedes further rotation of the retainer 600, until delivery of the second rod 702, as discussed below. The magnetic forces between the rods 700 may aid in aligning the rods 700 with one another during delivery, and may aid in retaining the rods 700 in contact with one another. Also as discussed, in some embodiments, regardless of whether the rods 700 are or are not magnets, the rods 700 may include features that engage with one another. Thus, for example, each of the rods 700, including the first rod 701 and the second rod 702, may include the features that engage with one another, such that when the first rod 701 and the second rod 702 contact one another, the rods 700 remain in contact with one another and do not separate from one another.

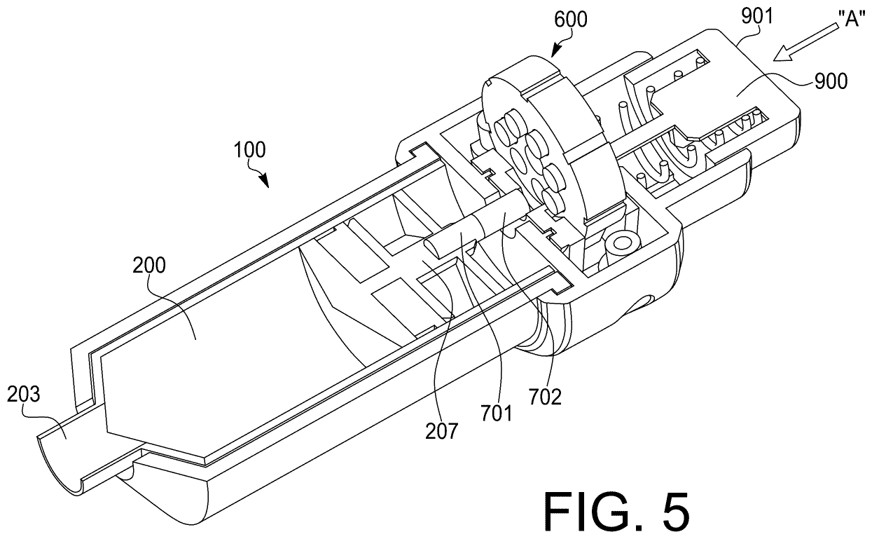

With reference to FIG. 5, the figure shows a third configuration in which the first dose of the medication has been delivered by the pump device 100 through the outlet 203 of the chamber 200. Specifically, a force has been applied against the pushing surface 901 of the button 900 in the direction A, which has moved the protrusion 905 in the direction A. With reference to FIGS. 2 and 5, the protrusion 905 is aligned with the opening 801 of the second plate 800, such that when the button 900 was pushed, the protrusion 905 contacted the second rod 702 within the rod opening 601 of the retainer 600 aligned with the opening 801 of the second plate 800. The protrusion 905 pushed the second rod 702 from the rod opening 601 of the retainer 600, through the opening 501 of the first plate 500 (which also is aligned with the opening 801 of the second plate 800 and the rod opening 601). The opening 501 of the first plate 500 aligned the second rod 702 with the first rod 701 previously received within the receiving portion 207 of the plunger 205, such that the two rods 700 are in contact with one another.

As discussed, in some embodiments, each of the rods 700 are magnets. Thus, in some embodiments, as the retainer 600 rotates on the axle 503 of the first plate 500, the attractive force between the first rod 701 already within the receiving portion 207 of the plunger 205 and the second rod 702 which is to be pushed into contact with the first rod 701 aligns the rods 700 with one another.

It is understood that a distance the plunger 205 moves within the chamber 200, and thus a volume of the dose of the medication that is delivered by pushing button 900, corresponds to lengths of the rods 700.

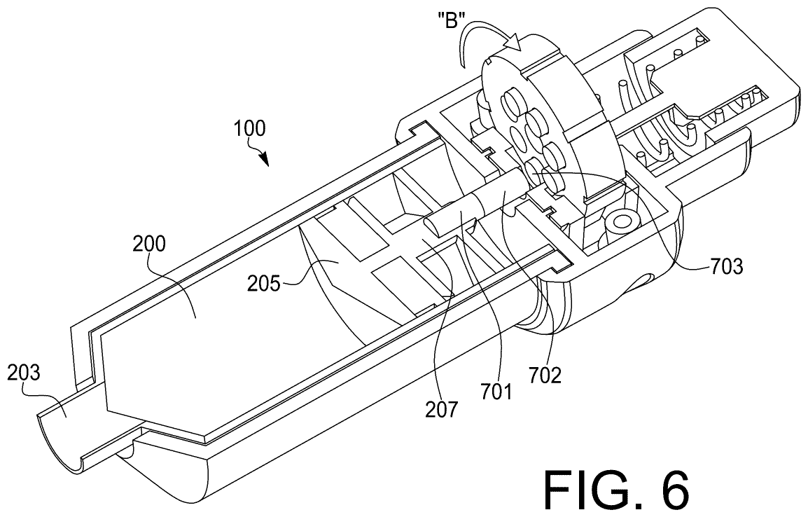

With reference to FIG. 6, the figure shows a fourth configuration in which the second dose of the medication has not yet been delivered by the pump device 100 through the outlet 203 of the chamber 200, but the pump device 100 is configured to deliver the second dose of the medication upon pushing of the button 900. As the figure shows, the retainer 600 has been rotated on the axle 503 of the first plate 500 (see, e.g., FIG. 2), in the direction B. Thus, after rotation, as the figure shows, another rod opening 601 of the retainer 600, in which a third one of the rods 700 (designated as third rod 703) is removably retained, is aligned with the opening 501 of the first plate 500.

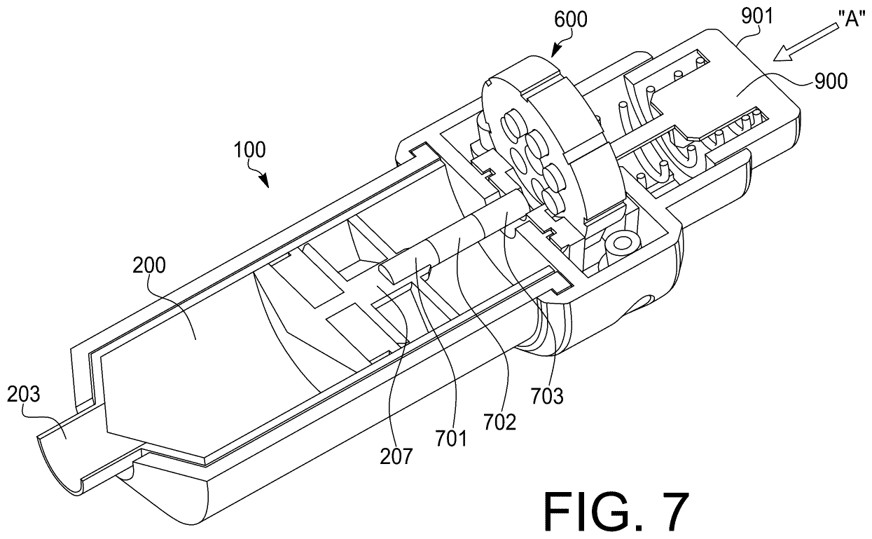

With reference to FIG. 7, the figure shows a fifth configuration in which the second dose of the medication has been delivered by the pump device 100 through the outlet 203 of the chamber 200. Specifically, a force has been applied against the pushing surface 901 of the button 900 in the direction A, which has moved the protrusion 905 (see, e.g., FIG. 2) in the direction A. With reference to FIGS. 2 and 7, the protrusion 905 is aligned with the opening 801 of the second plate 800, such that when the button 900 was pushed, the protrusion 905 contacted the third rod 703 within the rod opening 601 of the retainer 600 aligned with the opening 801 of the second plate 800. The protrusion 905 pushed the third rod 703 from the rod opening 601 of the retainer 600, through the opening 501 of the first plate 500 (which also is aligned with the opening 801 of the second plate 800 and the rod opening 601). The opening 501 aligned the third rod 703 with the first rod 701 and the second rod 702, such that the three rods 700 are in contact with one another.

It is understood that further rotation of retainer 600, and further actuation of button 900 to push rods 700 from the retainer 600 dispenses further doses of the medication from the chamber 200.

It will be understood that pump device 100 may be used to deliver any applicable medication. Such medication may be in liquid form, gel form, or another form. Such medication may include, for example, any applicable anesthetic, ropivacaine, bupivacaine, markain, lidocaine, dextrose, etc.

The foregoing provides description of exemplary embodiments. It will be understood, however, that the pump device 100 may include other embodiments, additional components, fewer components, or other components than those described above. For example, in some embodiments, the rods 700 are stacked in the retainer 600, such as above or alongside one another, and a retaining spring moves the rods 700 into the position from which one of the rods 700 may be delivered to the receiving portion 207 of the plunger 205, and from which the other rods 700 in the retainer may be delivered to contact and stack above one another, as described.

In another embodiment, lengths of the rods 700 may differ from one another, and the rods 700 may be disposed in the retainer 600 such that the doses delivered by the pump device 100 may vary from one another. Thus, for example, in some embodiments, the rods 700 delivered from the retainer 600 may become progressively shorter in length, such that each does of medication delivered by the pump device 100 is less than a preceding dose. Accordingly, any given rod of the rods 700 may be a different size or length in comparison to one or more other rods of the rods 700. In some embodiments, rather than the rods 700 being pushed out of the retainer 600, the rods 700 may be pulled or rotated out of the retainer 600, such as by a spring or other mechanism. In some embodiments, the rods 700 may be pushed out of the retainer 600 by one or more levers. In some embodiments, the rods may be stacked proximate to a syringe such that the rods are spring loaded to load a next rod after a given rod is used to perform a delivery of medication. In some embodiments, one or more surfaces of the pump device 100, including surfaces of the lower body portion 300 or the upper body portion 400, may include anti-slip or slip-resistant features.

In some embodiments, the pump device 100 incorporates a time strip, which indicates a time between doses of the medication delivered from the chamber 200. In some embodiments, the pump device 100 may include structures or mechanisms that control dosage, such as a dosage setting mechanism, or sensors that sense or otherwise control dosage to prevent over-and under-dosing. In some embodiments, the pump device 100 may include leak preventing mechanisms to prevent leakage of the medication from the chamber 200. In some embodiments, the pump device 100 may include a child lock to prevent a child or other individual from delivering the medication from the chamber 200. Such a child lock may be unlocked using a force, friction, pressure, rotation, or other applicable mechanism. In some embodiments, the pump device 100 may include an alarm which must be deactivated to prevent and/or suspend an audible or visible indication that medication has been delivered from the chamber 200.

In some embodiments, the pump device 100 is received at a pump holder (not shown). The pump holder may receive the pump device for storage purposes (e.g., in a vertical upright position, a horizontal position, an angled position, etc.). The pump holder may include or otherwise be associated with one more sensors which may be motion sensors, pressure sensors, hall effect sensors, electronic sensors, magnetic sensors, and/or the like. The pump holder include any applicable material such as foam, plastic, metal, elastomeric material, and/or the like or a combination thereof.

The one or more sensors may detect pump device 100 attributes such as an occurrence of pump device 100 removal/insertion to or from the holder, time of pump device 100 removal/insertion, dosage or use (e.g., a number or amounts of activations or deliveries of medication), pump device 100 use durations, pump device 100 medication properties, and/or the like.

As an example, the pump device holder may include one or more hall effect sensors which incorporate one or more Hall elements, each of which may produce a voltage proportional to a component of the magnetic field vector using the Hall effect. Specifically, the hall effect sensor(s) may detect a number of rods 700 deployed in the pump device 100 (e.g., prior to pump device 100 being retrieved from the pump device holder, when pump device 100 is retrieved from the pump device holder, or while the pump device 100 is positioned in the pump device holder). The pump device holder may determine pump related attributes such as an amount of medication in the pump device 100 (e.g., an initial amount, an amount detected after the pump device 100 is retrieved and repositioned in the pump device holder, etc.), an amount of medication expelled between the pump device 100 being retrieved and repositioned in the pump device holder, a number of rods 700 deployed, and/or the like. Alternatively, or in addition, the pump device holder may transmit sensed data (e.g., via wired or wireless signals) to an external component (e.g., a component including a processor) and the external component may determine the pump related attributes based on the received sensed data.

An action may be triggered based on the pump related attributes. The action may be triggered at the pump device holder, at pump device 100, at the external component, or at another external component. For example, a lockout period may be implemented based on sensing the time the pump device 100 is repositioned in the pump device holder, an amount of medication dispensed or number of rods 700 deployed between pump device 100 being retrieved and repositioned in the pump device holder, a lockout timer or instruction (e.g., a processor instruction for a processor included in or associated with the pump device holder), and/or the like. The lockout period may be implemented by triggering a locking mechanism at the pump device holder and/or pump device 100 that locks the pump device 100 from being retrieved from the pump device holder. The duration of the lockout period may be pre-determined or may be determined based on one or more of the pump related properties.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

All of the aspects described in the present disclosure (including references incorporated by reference, accompanying claims, abstract and drawings), may be combined in any order, in part or in full, or in any combination or modification, except when such are incompatible or inconsistent. Furthermore, each aspect may be replaced by alternative features serving the same, equivalent or similar purpose, unless expressly stated otherwise or inconsistent with the teachings herein. Thus, unless expressly stated otherwise, each aspect disclosed herein may be only an example of equivalent or similar features. It is intended that the invention be defined by the attached claims and their legal equivalents.

What is claimed is:

1. A pump device configured to deliver a medication, comprising:
a body portion;
a chamber disposed in the body portion, the chamber including an interior and an outlet, the interior configured to hold the medication and to deliver the medication through the outlet;
a plunger disposed in the chamber and configured to move within the interior of the chamber to deliver the medication in the interior through the outlet, the plunger including a receiving portion;
a retainer disposed within the body portion, the retainer including a plurality of rod openings;
a plurality of rods removably retained within the plurality of rod openings; and
a button disposed in the body portion, the button including a pushing surface and a protrusion, the button configured, when the pushing surface of the button is pushed, to move within the body portion such that the protrusion of the button is brought into contact with a first rod of the plurality of rods in a first rod opening in the plurality of rod openings and to move the first rod out of the first rod opening and into the receiving portion of the plunger.

2. The pump device of claim 1, wherein the retainer is configured to rotate to align a second rod of the plurality of rods in a second rod opening of the plurality of rod openings with the first rod in the receiving portion of the plunger.

3. The pump device of claim 1, wherein the retainer is configured to rotate to align a second rod of the plurality of rods in a second rod opening of the plurality of rod openings with the first rod in the receiving portion of the plunger,
wherein the button is configured, when the pushing surface of the button is pushed, to move within the body portion such that the protrusion of the button is brought into contact with the second rod and to move the second rod out of the second rod opening and into the receiving portion of the plunger, thereby delivery a first dose of the medication from the interior of the chamber through the outlet of the chamber.

4. The pump device of claim 1, wherein the body portion includes an upper body portion and a lower body portion coupled to one another,
wherein the chamber is disposed within the lower body portion, and
wherein the button is disposed in the upper body portion.

5. The pump device of claim 1, wherein the retainer is configured to rotate in only one direction.

6. The pump device of claim 1, wherein at least one of the protrusion of the button and the plurality of rods comprises a magnet, and the other one of the protrusion and the plurality of rods comprises a magnetic material, such that the protrusion and one rod of the plurality of rods are magnetically attracted to one another when the one rod of the plurality of rods is aligned with the protrusion.

7. The pump device of claim 1, wherein the protrusion of the button is configured to slide one rod of the plurality of rods from the retainer into a receiving portion of the plunger.

8. The pump device of claim 1, wherein the pump device has a bottom end and a top end opposite the bottom end, wherein the chamber is disposed closer to the bottom end than the top end, and
wherein the retainer is configured to rotate in a clockwise direction, when viewed from the top end of the pump device, to align a second rod of the plurality of rods in a second rod opening of the plurality of rod openings with the first rod in the receiving portion of the plunger.

9. A pump device configured to deliver a medication, comprising:
a body portion;
a chamber disposed in the body portion, the chamber including an interior and an outlet, the interior configured to hold the medication and to deliver the medication through the outlet;
a plunger disposed in the chamber and configured to move within the interior of the chamber to deliver the medication in the interior through the outlet, the plunger including a receiving portion;
a retainer disposed within the body portion;
a plurality of rods removably retained within the retainer; and
a mechanism configured to move the plunger within the chamber thereby delivery a dose of medication from the chamber, wherein the mechanism is configured to engage with at least one rod of the plurality of rods, thereby moving the plunger within the chamber.

10. The pump device of claim 9, wherein the retainer is configured to rotate to align a second rod of the plurality of rods in a second rod opening in the retainer with a first rod in the receiving portion of the plunger.

11. The pump device of claim 9, wherein the retainer is configured to rotate to align a second rod of the plurality of rods in a second rod opening in the retainer with a first rod in the receiving portion of the plunger,
wherein the pump device further comprises a pushing surface configured to move within the body portion such that a protrusion is brought into contact with the second rod and to move the second rod out of the second rod opening and into the receiving portion of the plunger, thereby delivery a first dose of the medication from the interior of the chamber through the outlet of the chamber.

12. The pump device of claim 9, wherein the body portion includes an upper body portion and a lower body portion coupled to one another,
wherein the chamber is disposed within the lower body portion.

13. The pump device of claim 9, wherein the retainer is configured to rotate in only one direction.

14. The pump device of claim 9, wherein the plurality of rods comprises a magnet, or a magnetic material.

15. The pump device of claim 9, further comprising a button configured to slide one rod of the plurality of rods from the retainer into a receiving portion of the plunger.

16. The pump device of claim 9, wherein the pump device has a bottom end and a top end opposite the bottom end, wherein the chamber is disposed closer to the bottom end than the top end, and
wherein the retainer is configured to rotate in a clockwise direction, when viewed from the top end of the pump device, to align a second rod of the plurality of rods in a second rod opening in the retainer with a first rod in the receiving portion of the plunger.

17. A method of delivering a dose of medication with a pump device including a body portion, a chamber disposed in the body portion, the chamber including an interior and an outlet, the interior holding the medication and configured to deliver the medication through the outlet, a plunger disposed in the chamber and configured to move within the interior of the chamber to deliver the medication in the interior through the outlet, the plunger including a receiving portion, a retainer disposed within the body portion, the retainer including a plurality of rod openings, a plurality of rods removably retained within the plurality of rod openings, and a button disposed in the body portion, the button including a pushing surface and a protrusion, the method comprising:
pushing the pushing surface of the button such that the protrusion of the button is brought into contact with a first rod of the plurality of rods in a first rod opening in the plurality of rod openings, to thereby move the first rod out of the first rod opening and into the receiving portion of the plunger; and
delivering a first dose of medication through the outlet of the body portion, wherein a volume of the first dose corresponds to a length of the first rod.

18. The method of claim 17, further comprising:
advancing the retainer such that a second rod in a second rod opening of the plurality of rod openings is aligned with the protrusion.

19. The method of claim 17, further comprising:
advancing the retainer such that a second rod in a second rod opening of the plurality of rod openings is aligned with the protrusion;
pushing the pushing surface of the button such that the protrusion of the button is brought into contact with the second rod in the second rod opening, to thereby move the second rod out of the second rod opening and into the receiving portion of the plunger; and
delivering a second dose of medication through the outlet of the body portion, wherein a volume of the second dose corresponds to a length of the second rod.

20. The method of claim 17, further comprising prior pushing the pushing surface, advancing the retainer such that first rod is aligned with the protrusion.
